(19) United States
(12) Patent Application Publication
(10) Pub. No.: US 2024/0310253 A1
(43) Pub. Date: Sep. 19, 2024

(54) SELF-USE SAMPLE COLLECTION KIT AND INTEGRATED APPLICATION AND SYSTEMS THEREOF

(71) Applicant: LEDA HEALTH CORPORATION, Brooklyn, NY (US)

(72) Inventors: Madison P. CAMPBELL, Brooklyn, NY (US); Liesel VAIDYA, Brooklyn, NY (US)

(21) Appl. No.: 18/580,128

(22) PCT Filed: Aug. 16, 2022
(86) PCT No.: PCT/US2022/040479

§ 371 Date: Jan. 17, 2024

Related U.S. Application Data
(60) Provisional application No. 63/234,229, filed on Aug. 17, 2021.

(51) Int. Cl. G01N 1/10

(52) U.S. Cl. CPC G01N 1/10; G01N 2001/1056

(57) ABSTRACT

An integrated self-use sample collection kit and system designed for the collection and tracking of DNA and other bodily fluids that meets legal and forensic evidentiary standards so it can be used, for example, at a criminal trial, civil trial, or other legal proceeding.

This application claims the priority benefit of U.S. Provisional Patent Application Ser. No. 63/234,229, filed Aug. 17, 2021, which is hereby incorporated by reference in its entirety.

### FIELD

The present disclosure relates to a self-use sample collection kit and integrated application. More particularly, the disclosure relates to a sample collection kit that (1) allows an individual to collect, store, secure, and preserve evidence herself/himself, without in-person assistance; and (2) meets applicable legal and forensic standards. Further, the integrated application allows for customized guidance and instructions for collection, as well as electronic collecting, tagging, organizing, and tracking of the evidence for later use in law enforcement, judicial proceedings, or military operations, for example.

### BACKGROUND

Personal use sample collection kits have been developed for use in DNA testing. These collection kits allow a user to collect, store, and preserve organic samples herself/himself, without in-person assistance. However, these types of kits are generally limited in the type of samples collected and do not provide security necessary where the possibility of tampering is a legal issue. There is an unmet need for sample collection systems and methods to ensure the self-collected biological material meets evidentiary standards. Additionally, available collection kits do not meet applicable legal and forensic standards required for collecting samples related to a crime or tort, such as a sexual assault, and are therefore unsuited for collection of evidentiary material.

Traditional hospital sexual assault examination kits (SAEKs) have been unchanged for decades and are antiquated. Current SAEKSs generally require that the collection of evidence be performed at a hospital by a specially-trained sexual assault nurse examiner (SANE). This is required mainly due to the lack of a product or service that assures that evidence can be properly collected without the physical presence of a third party, the SANE, and that the evidence will not be tampered with. A significant number of evidentiary rules exist to attempt to prevent the presentation of irrelevant, unreliable, or false evidence. As the evidence is often the basis for a legal determination of guilt or innocence in a criminal proceeding, or liability in a civil proceeding, if the underlying evidence is flawed, inaccurate, or presented inaccurately, an erroneous determination can be made. It is therefore necessary to make sure that evidentiary rules are followed to make sure that what is presented at trial is ultimately the same as what was originally collected.

There is a need for more effective evidence collection systems and methods to address and analyze criminal and tort situations. For example, after an assault, the victim is often traumatized. The step of collecting the required evidence must be done at a time when the victim is at his/her most vulnerable state. Requiring the victim to be present at a hospital may preclude many victims from coming forward as they are often afraid or ashamed, therefore being uncomfortable undergoing a formal forensic exam. Prior to the forensic exam, the victim must avoid showering, bathing, or changing her/his clothes, which further disincentivizes a traumatized victim from reporting the assault to law enforcement.

As a further obstacle to traumatized victims, many hospitals lack the capacity to administer the forensic exam. According to the International Association of Forensic Nurses, only about 17-20% of hospitals in the United States have SANEs on staff. In less developed countries, access to sexual assault kits is even more restricted. Therefore, it is evident that a collection device, with systems and methods thereof that meets applicable legal and forensic standards while allowing a victim to collect the evidence in a more intimate setting, such as at the victim's home, is direly needed. Additionally, there is a need for an integrated system that provides guidance and instructions to encourage collection and ensure that collection is properly performed to preserve evidence.

It is understood that there are many other potential uses and needs for improved evidence collection systems and methods, including integrated applications to assist in collection.

The present technology is directed to these and other deficiencies in the art.

### SUMMARY

Because of these and other reasons, there are described herein a sample collection kit, an associated application provision system integrated with the sample collection kit that provides for electronic monitoring and tracking of evidence and electronic storage of investigatory materials collected using the sample collection kit. The systems relate to electronic systems for identifying, storing, and evaluating evidence. The systems also utilize a combination of computer readable storage of investigatory material, along with computer storage of evidence identification, with the use of smart contracts in a blockchain to ensure integrity of the data.

The technology is useful for collection of evidence that meets applicable forensic and evidentiary standards. In some embodiments the technology is useful for self-directed collection of evidence.

There are many advantages of the disclosed sample collection kit and integrated application provision system, as will be further appreciated from the discussions herein. For example, the sample collection device can be used without the need to be at a hospital in the physical presence of a SANE, reducing the chances of unreported incidents. The design of the sample collection kit allows for the samples to be collected and secured in a tamper-evident and tamper-resistant manner, which ensures applicable forensic and evidentiary standards are met. The tracking and electronic storage systems and methods ensure that the evidence is always accounted for, while the usage of smart contracts within a blockchain ensures that the data cannot be tampered with.

In accordance with one aspect of this disclosure, provided herein is a sample collection kit. In one embodiment, the sample collection container includes: (a) a first portion having a distal end and a proximal end, wherein the distal end comprises a swab configured to collect sample; (b) a second portion having an enclosure configured to receive at least the distal end of the first portion, wherein the sample collection container forms a tamper element when the first portion and the second portion are coupled together thereby isolating the swab; and (c) a unique identifier label affixed to at least a portion of the sample collection container, with the unique identifier label uniquely identifying the sample collection container from a population of sample collection containers. The population of sample collection containers includes at least one, and in some instances many, sample collection containers.

In accordance with another aspect of this disclosure, provided herein is an evidence collection kit including a sample collection container. In one embodiment, the sample collection container includes: (a) a first portion having a distal end and a proximal end, wherein the distal end comprises a swab configured to collect sample; (b) a second portion having an enclosure configured to receive at least the distal end of the first portion, wherein the sample collection container forms a tamper element when the first portion and the second portion are coupled together thereby isolating the swab; and (c) a unique identifier label affixed to at least a portion of the sample collection container, with the unique identifier label uniquely identifying the sample collection container from a population of sample collection containers.

In some embodiments, the above-discussed container may include one or more of the following features either individually or in combination with other features. For example, in some embodiments the tamper element may include at least one of: a seal, a lock, a tape, a threaded element, or a microchip. Additionally, the tamper element may include at least one of: a tamper-proof element, a tamper resistant element, and a tamper evident element.

Further, in some embodiments, the above-discussed container may include: a purifying element configured to prevent or reduce contamination of the swab. Additionally, in some embodiments, the purifying element may include at least one of: a seal, a lock, a tape, a threaded element, or any combination thereof. In some embodiments, the anti-tamper element is the purifying element.

The above-mentioned container, in some embodiments, may include a swab that is substantially free of DNA. Additionally, in some embodiments, the swab is a dry swab. Also, in some embodiments, the swab is sterile. Further, in some embodiments, the swab may comprise: a natural fiber, a polymer fiber, or a combination thereof. Also, in some embodiments, the swab comprises flocked fibers.

In some embodiments, the above-mentioned container may include: a unique identifier label that is attached to the one or more elements of the sample collection kit. Additionally, in some embodiments, the unique identifier label may comprise: a barcode, an RFID element, a QRS code, or a combination thereof.

In some embodiments, the one or more elements of the sample collection kit include a unique identifier label that is configured to communicate with a digital platform or remote user. The unique identifier label may also be configured to store data. Further, the data may include: a personally-identifying information, a medical history, a date, a location, or any combination thereof. In some examples, the data may include photographs, testimonial evidence from the sexual assault victim, and witness testimony from the SANE, although other data may be contemplated. Because the data stored relates to medical/health information, the present technology is configured to be HIPAA compliant in some examples.

In some embodiments, a kit may include more than one of the above-mentioned sample collection containers. The above-mentioned kit, in some embodiments, may include: a substrate, a sample bag, a glove, an aqueous solution, or any combination thereof. Further, in some embodiments, the kit may include an aqueous solution wherein the aqueous solution comprises distilled water, substantially free from DNA. Additionally, in some embodiments, the above-mentioned kit may include a copy of the unique identifier label, wherein the copy comprises an adhesive backing.

In accordance with a further aspect of this disclosure, provided herein is a method of making the above-discussed evidence collection kit. In some embodiments, the evidence collection kit may be assembled by: (a) including a tamper-resistant, or tamper-evident, outer container that may comprise: (i) a cardboard box sealed by tamper-evident adhesive label; (ii) plastic container sealed by tamper-evident mechanism; or (iii) any other type of tamper-evident container; (b) one of more tamper-evident evidence collection containers that are included inside the above-mentioned tamper-evident outer container; and (c) an instructions manual.

In accordance with another aspect of this disclosure, provided herein is a computer-implemented sample collection platform. In one embodiment, the computer-implemented forensic evidence collection platform includes: (a) a central processor configured to provide a central application assigning an identification code to one of the plurality of kits; (b) a kit processor coupled to a positioning system and performing at least the following: (i) receiving the identification code; (ii) recording a kit location via the positioning system; (iii) transmitting the kit location and the identification code to the central processor; (iv) asynchronously creating a smart contract with kit location and timestamp; and (v) transmitting smart contract address and Tx hash to central processor.

The evidence collection platform also includes (c) a mobile processor configured to provide a mobile application performing at least the following: (i) recording the identification code; (ii) recording a victim attribute and witness attribute; (iii) recording a sample collection time and at least one of a descriptor or an image, wherein the sample collection time, the descriptor, the image, or any combination thereof are associated with an evidence specimen deposited in a container within a container within the one of the plurality of kits containing the identification code; (iv) transmitting the identification code, the victim attribute, the witness attribute, the sample collection time, and at least one of the descriptor or an image to the central processor; (v) receiving a current status of the container from the central processor; (vi) displaying the current status; (vii) saving the identification code, the victim attribute, the witness attribute and the sample collection time to the smart contract; and (viii) transmitting Tx hash from blockchain to central processor.

In some embodiments, the above-mentioned platform comprises a central application that may: confirm identification code received from the kit processor, the mobile application, or both. Further, in some embodiments, the identification code may include: a numeric code, a letter-based code, an alphanumeric code, a bar code, a QR code, an image, or any combination thereof. Additionally, in some embodiments, the identification code may include: a kit identification code, a container identification code, or both.

The above-mentioned platform, in some embodiments, may comprise a victim attribute, wherein it may include: a name, an address, an age, a sex, a signature, a driver's license number, a social security number, an image, or any combination thereof. Additionally, in some embodiments, the platform may comprise a witness attribute, wherein it may include: a name, an address, an age, a sex, a signature, a driver's license number, a social security number, an image, or any combination thereof.

In some embodiments, the above-mentioned platform may send the kit location, and receive the current status in real-time. Further, in some embodiments, the recording of the kit location only occurs after the transmission of the identification code, the victim attribute, the witness attribute, the sample collection time, and at least one of the descriptors or an image to the central processor. Additionally, in some embodiments, the kit location, the sample collection time, the identification code, the victim attribute and the witness attribute may be recorded in the smart contract in the blockchain. Also, in some embodiments, the kit location, the identification code, the victim attribute, the sample collection time, the descriptor, or the image may be encrypted before transmission.

In some embodiments, the above-mentioned platform may record the identification code, the victim attribute, the witness attribute, the sample collection time or the descriptor is performed via telemedicine.

In accordance with a further aspect of this disclosure, provided herein is a computer-implemented forensic evidence collection method comprising: (a) providing a central application assigning an identification code to one of the plurality of kits using a central processor; (b) providing a kit processor coupled to a positioning system, the kit processor performing at least the following: (i) receiving the identification code; (ii) recording a kit location via the positioning system; (iii) transmitting the kit location and the identification code to the central processor; (iv) asynchronously creating a smart contract with kit location and timestamp; and (v) transmitting smart contract address and Tx hash to central processor; and (c) a mobile processor configured to provide a mobile application performing at least the following: (i) recording the identification code; (ii) recording a victim attribute and witness attribute; (iii) recording a sample collection time and at least one of a descriptor or an image, wherein the sample collection time, the descriptor, the image, or any combination thereof are associated with an evidence specimen deposited in a container within a container within the one of the plurality of kits containing the identification code; (iv) transmitting the identification code, the victim attribute, the witness attribute, the sample collection time, and at least one of the descriptor or an image to the central processor; (v) receiving a current status of the container from the central processor; (vi) displaying the current status; (vii) saving the identification code, the victim attribute, the witness attribute and the sample collection time to the smart contract; and (iix) transmitting Tx hash from blockchain to central processor.

In some embodiments, the above-mentioned method may confirm, by the central processor, the identification code received from the kit processor, the mobile application, or both. Further, in some embodiments, the identification code may include, for example: a numeric code, a letter-based code, an alphanumeric code, a bar code, a QR code, an image, or any combination thereof. Additionally, the identification code may comprise a kit identification code, a container identification code, or both.

The above-mentioned method, in some embodiments, may comprise a victim attribute, wherein it may include: a name, an address, an age, a sex, a signature, a driver's license number, a social security number, an image, or any combination thereof. Further, the method, in some embodiments, may comprise a witness attribute, wherein it may include: a name, an address, an age, a sex, a signature, a driver's license number, a social security number, an image, or any combination thereof.

In some embodiments, the above-mentioned method may send the kit location, and receive the current status in real-time. Further, in some embodiments, the recording of the kit location only occurs after the transmission of the identification code, the victim attribute, the witness attribute, the sample collection time, and at least one of the descriptor or an image to the central processor. Additionally, in some embodiments, the kit location, the sample collection time, the identification code, the victim attribute and the witness attribute may be recorded in the smart contract in the blockchain. Also, in some embodiments, the kit location, the identification code, the victim attribute, the sample collection time, the descriptors, or the image may be encrypted before transmission.

In some embodiments, the above-mentioned method may record the identification code, the victim attribute, the witness attribute, the sample collection time or the descriptor is performed via telemedicine.

It is understood that there are many other features and advantages associated with the disclosed systems and methods, as will be appreciated from the discussions below.

### BRIEF DESCRIPTION OF THE DRAWINGS

The foregoing features of the disclosure, as well as the disclosure itself may be more fully understood from the following detailed description of the drawings, in which:

FIG. 1 illustrates a parts list for an exemplary sample collection kit of the present technology.

FIG. 2 illustrates a set of bar codes that may be employed on items in the sample collection kit to provide unique identification of those items.

FIG. 3 shows a non-limiting block diagram of a computer system for providing an integrated application for use with the sample collection kit of FIG. 1;

FIG. 4A shows a non-limiting block diagram example of a web/mobile application provision system;

FIG. 4B shows a non-limiting block-diagram example of a smart contract and blockchain system;

FIG. 5 shows a non-limiting block diagram example of a cloud-based system;

FIG. 6 shows an exemplary signup flow for the integrated sample collection kit and application of the present technology.

FIG. 7 shows an exemplary login flow for the integrated sample collection kit and application of the present technology.

FIG. 8 shows an exemplary registration flow for the integrated sample collection kit and application of the present technology.

FIG. 9 shows an exemplary incident reporting flow for the integrated sample collection kit and application of the present technology.

FIG. 10 shown an exemplary image upload flow for the integrated sample collection kit and application of the present technology.

### DETAILED DESCRIPTION

The present technology provides an integrated self-use sample collection kit and application, and methods of use thereof for collecting and processing biological samples and other materials. The sample collection kit may be employed, for example, in collecting evidence of crimes or torts including, but not limited to, sexual assault that meets evidentiary and forensic standards, although any other uses that involve the secure collection and management of samples is envisioned. The integrated sample collection kit and application advantageously allow for self-use of the collection kit, which allows a victim to collect evidence at a more intimate and accessible location, which should increase collection and reporting of such crimes or torts. Further the integrated application provides for support, guidance, and instructions related to the use of the sample collection kit.

The sample collection kit is intended to be employed in any situation in which a victim is unable or unwilling to have a prompt in-person examination by a SANE, or in which a SANE is unable or unwilling to provide an in-person examination of a victim. For example, the sample collection kit described herein may be used in a variety of settings, including but not limited to colleges and universities, military installations, prisons, refugee camps, and large outdoor gatherings like music festivals, as well as hospitals, clinics, and other healthcare facilities. Further, the integration between the sample collection kit and the application allows for registration of the kit, providing information regarding the use of the kit to users, secure handling of sensitive information, and maintaining a chain of custody, as well as other advantages described herein.

The sample collection kit of the present technology provides a tamper-resistant kit that allows a survivor of sexual assault to collect, store, secure, and preserve evidence privately in a safe setting. All evidence collected can be stored indefinitely at room temperature. The integrated application collects necessary personal data, guides a survivor through the evidence collection process using an instructional video, provides an option to contact a designated person for support, generates documentation for criminal, civil, or other legal proceedings, and provides storage instructions. The application is end-to-end encrypted, provides for tracking and utilizes blockchain to create immutable timestamps and other records.

The combined sample collection kit and integrated application are advantageously private, easy-to-use, and accessible while meeting applicable legal and forensic standards.

It is understood that any specific embodiments described herein are shown by way of illustration and not as limitations of the disclosure and the concepts described herein. Features of the subject matter described herein can be employed in various embodiments without departing from the scope of the concepts sought to be protected.

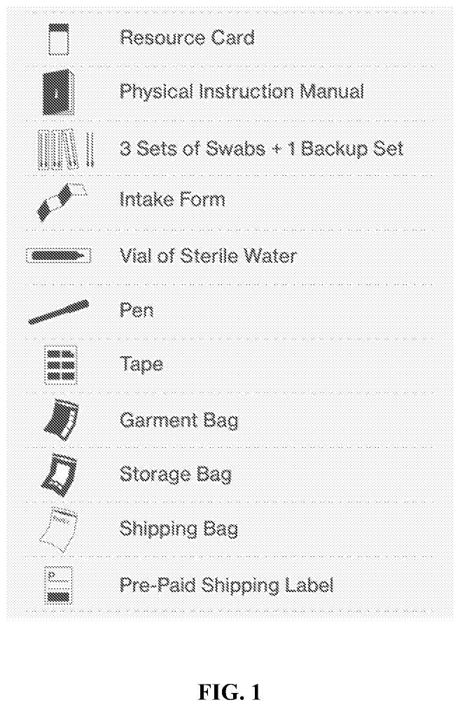

FIG. 1 illustrates a parts list for an exemplary sample collection kit of the present technology. The sample collection kit described herein contains various materials for preserving and collecting forensic evidence, by way of example, as well as materials to facilitate the collection of samples, to integrate the sample collection kit with the integrated application as described in further detail below, and to provide support to the user. In this example, the sample collection kit includes a resource card, a physical instruction manual, swabs, an intake form, a vial of sterile water, a pen, tape, a garment bag, a storage bag, a shipping bag, and a pre-paid postage label, although the sample collection kit may include other types and/or number of items in other combinations.

In this example, the sample collection kit is provided in a packaging that includes an outer cardboard box that houses each of the items in the sample collection kit and allows for convenient storage of the sample collection kit. The cardboard box also houses and contains three smaller boxes for collection of sample materials as described in further detail below, although other numbers of smaller boxes may be employed. In particular, the smaller cardboard boxes are configured to receive swabs used to collect materials and include vent holes to facilitate air-drying of samples collected using the swabs.

The sample collection kit includes a resource card that identifies resources available to, for example, sexual assault victims. The resource card may provide various types of information and is not limited in the information that may be included thereon.

The physical instruction manual includes instruction on the use of the sample collection kit, although the user may receive additional instructions and guidance through the integrated application as described further below.

In this example, the sample collection kit includes three sets of swabs and an additional backup set of swabs, although the sample collection kit may include any number of swabs. The swabs are sterile and cotton-tipped and may be employed to collect, for example, DNA in saliva (the survivor's reference sample) and semen (the perpetrator's sample), although the swabs may be employed to collect other biologic materials like hair, skin, dried blood, dried urine, or any other types of samples. In one example, the swabs are 6″ “Cap-Shure” swabs produced by Puritan Medical Products (Guilford, ME), although other types and/or sizes of swabs may be utilized.

The sample collection kit includes an intake form that is used to collect information from the user, although information may also be collected from the user through the integrated application as described herein.

The sample collection kit also includes a vial of sterile water that is used to prevent contamination. In one example, the vial contains 3 milliliters of sterile water, although other sizes may be employed. The sample collection kit may also include other solutions, such as used for sterilization of equipment or processing collected samples. Solutions can include, for example, aqueous solutions, stabilizing solutions, buffers, distilled water, and sterilizing solutions. Stabilizing solutions can preserve the integrity of the collected biological material by reducing the growth of contaminating microbes and resisting extreme temperature changes during transport.

A pen is included for ease of use to fill out the intake form, by way of example.

The sample collection kit includes tamper-evident tape that may be employed to seal materials, such as the sample collection boxes, by way of example, to preserve integrity of the collected samples. Once opened, the tamper-evident tape will display a message: “Open and/or Void” that indicates the sealed material has been opened. The sealed tape indicates that that the samples have remained sealed. In one example, the tape is tamper-evident tape made by Tritech Forensics, Inc. (Raleigh, NC), although other types of tamper evident tapes may be employed.

The sample collection kit includes a garment bag configured to receive and store garments, such as the victim's clothing, by way of example. In this example, the garment bag is self-sealing, which allows for the garment bag to remain sealed to ensure integrity and chain of custody. The garment bag in one example is made of Tyvek®, which facilitates air-drying of any samples found on garments, although other suitable materials may be employed.

The sample collection kit includes a storage bag that is sized and configured to receive the entire box, including the sample collection boxes, and the garment bag once all samples have been collected. The storage bag is self-sealing, which allows for the storage bag to remain sealed to ensure integrity and chain of custody. The storage bag in one example is made of Tyvek®, which facilitates air-drying of the collected samples, although other suitable materials may be employed.

A shipping bag is included that is sized to receive the storage bag, including the collected samples, therein. In one example, the shipping bag is a FedEx® Clinical Pak that is specifically employed for shipping clinical samples, although other shipping materials may be employed. The sample collection kit also includes a pre-paid postage label to allow for shipping the materials to a pre-determined destination, such as a testing laboratory.

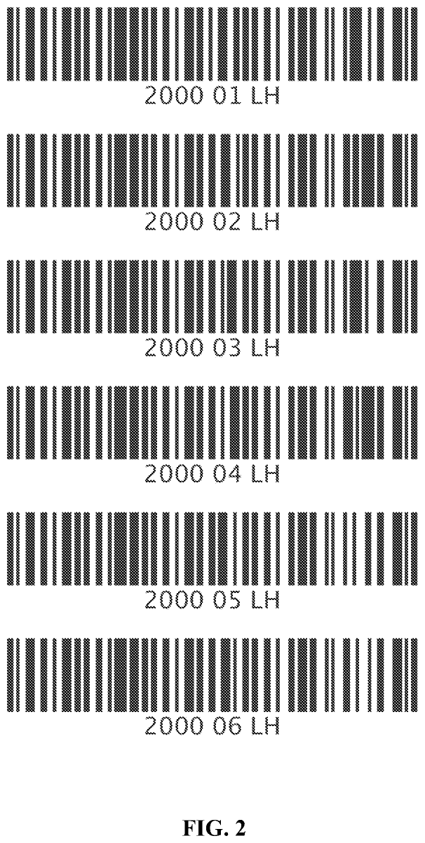

One or more items in the sample collection kit may include unique identifiers that are employed to allow the items to be registered in the integrated application as described further below. In this example, unique barcodes are employed although other identifiers such as a numeric code, a letter-based code, an alphanumeric code, a QR code, an image, or any combination thereof, may be employed. By way of example, FIG. 2 illustrates a series of “128” barcodes that serve as the interface between: (1) components of the physical sample collection kit; and (2) a user portal, online application, and a laboratory portal as described further below. In this example, the first 4 digits (2000) denote the kit number, the second 2 denote the component number (00-06), and the “LH” suffix denotes the company, although other identifiers may be employed. The number of digits for the kit number can increase, to accommodate more than 9999 kits or, if the first kit number is 2000, more than 8000 kits.

In one example, the barcodes are located on stickers that are affixed to the outer/kit box, each of the three swab collection boxes, and the garment bag, for example. Although the barcodes as shown in FIG. 2 are organized in series of 6 (2000 01, 2000 02, 2000 03, 2000 04, 2000 05, 2000 06), only the first 5 bar codes are used for each kit and its components in one example. A survivor of sexual assault, or other crime for which collection is desired, scans the bar code to access the online application, and enters the numbers and letters on the code to access the user portal, as described in further detail below. In addition, the testing laboratory enters the numbers and letters into the laboratory portal. In either case, the use of the unique identifiers allows for unique identification of the particular sample collection kit employed.

Further provided herein are methods of using the exemplary sample collection kit disclosed herein. A user of the sample collection kit can collect samples and assemble the sample collection kit in the packaging material. The user can further interact with a user portal provided in an integrated application as described in further detail below.

Although an example of the sample collection kit is described above, it is to be understood that the sample collection kit could include other types and/or numbers of materials in other combinations. As described below, the sample collection kit is configured to be employed with an integrated application disclosed herein that provides both a user portal and a laboratory portal for improved collection and processing of samples.

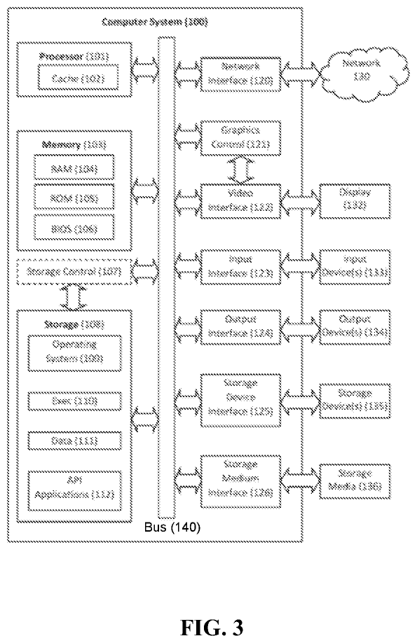

FIG. 3 is a block diagram showing an exemplary machine that includes a computer system 100 within which a set of instructions can execute for causing a device to perform or execute any one or more of the aspects and/or methodologies for providing an integrated application that is operable with the evidence collection kit of the present disclosure. The components in FIG. 3 are only examples and do not limit the scope of use or functionality of any hardware, software, embedded logic components, or any combination thereof implementing particular embodiments. In some embodiments, the computer system 100 may be employed in a virtual, cloud-computing network.

In some embodiments, computer system 100 may include one or more processors 101, a memory 103, and a storage 108 that communicate with each other, and with other components, via a bus 140. The bus 140 may also link a display 132, one or more input devices 133, one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices, laptop or notebook computers, distributed computer systems, computing grids, or servers.

In some embodiments, computer system 100 may include one or more processors 101 (e.g., central processing units (CPUs) or general-purpose graphics processing units (GPGPUs)) that carry out functions. Processor(s) 101 optionally contains a cache memory unit 102 for temporal local storage of instructions, data, or computer addresses. Processor(s) 101 are configured to assist in execution of computer readable instructions. Computer system 100 may provide functionality for the components depicted in FIG. 3 as a result of the processor(s) 101 executing non-transitory, processor-executable instructions embodied in one or more tangible computer-readable storage media, such as memory 103, storage 108, storage device(s) 135, and/or storage media 136. The computer-readable media may store software that implements particular embodiments, and processor(s) 101 may execute the software. Memory 103 may read the software form one or more other computer-readable media (such as mass storage devices 135, 136) or form one or more sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

In some embodiments, the memory 103 may include various components including, but not limited to, a random access memory (RAM) 104 (e.g., static RAM (SRAM), dynamic RAM (DRAM), ferroelectric RAM (FRAM), phase-change RAM (PRAM), etc.), a read-only memory component (ROM) 105, and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system (BIOS) 106, including basic routines that help to transfer information between elements within computer system 100, such as during start-up, may be stored in the memory 103.

In some embodiments, fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, application programs 112, and the like. Storage 108 can also include an optional disk drive, a solid-state device, or any combination thereof. Information in storage 108 may be incorporated as virtual memory in memory 103, when appropriate.

In one example, storage devices 135 may be removably interfaced with computer system 100 via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

In some embodiments, bus 140 connects a wide variety of subsystems. Herein, reference to a bus may encompass one or more digital signal lines serving a common function. Bus 140 may be any of several types of bus structures including, but not limited to, a memory bus, a memory controller, a peripheral bus, a local bus, and any combination thereof, using any variety of bus architectures. As an example such architectures may include, but not limited to, an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, Hyper Transport (HTX) bus, serial advanced technology attachment (SATA) bus, and any combination thereof.

Computer system 100 may also include an input device 133. In some embodiments, the input device is a Kinect, Leap Motion, or the like. Input device(s) 133 may be interface to bus 140 via any of a variety of input interfaces 123 including, but not limited to, serial, parallel, game port, USB, Firewire, thunderbolt, or any combination of the above.

In some embodiments, when computer system 100 is connected to network 130, computer system 100 may communicate with other devices, especially mobile devices and enterprise systems, distributed computing systems, cloud storage systems, cloud computing systems, and the like, connected to network 130. Communications to and from computer system 100 may be sent through network interface 120. In one example, computer system 100 communicates with a user computing device, which may be for example a mobile computing device, that allows for integration between a sample collection kit (as described above) employed by the user and an application therefore, as described below. In another example, computer system 100 communicates with a computing device operated by a laboratory that provides information regarding testing of samples collected using the exemplary sample collection kit described above, although the computer system 100 may communicate with other devices, including other storage devices, such as databases, by way of example.

Information and data can be displayed through a display 132. The display 132 can interface with the processor(s) 101, memory 103, and fixed storage 108, as well as other devices, such as input devices(s) 133, via the bus 140. The display 132 is linked to the bus 140 via a video interface 122, and transport of data between the display 132 and the bus 140 can be controlled via the graphics control 121.

Those of skill in the art will appreciate that the various illustrative logical blocks, modules, circuits, and algorithm steps described in connection with the embodiments disclosed herein may be implemented as electronic hardware, computer software, or combinations of both.

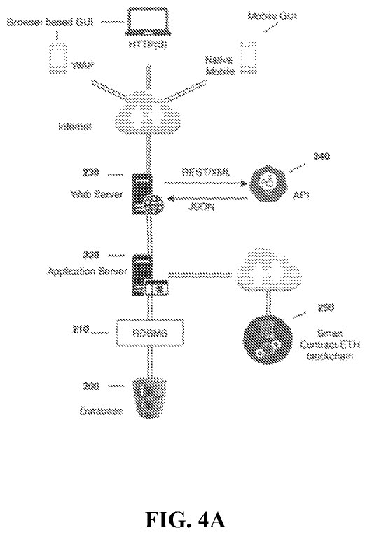

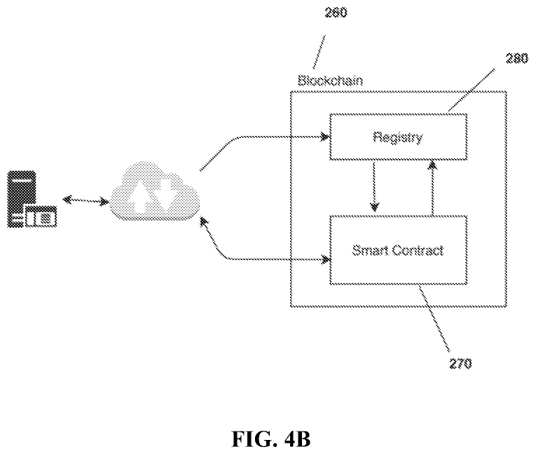

Referring to FIG. 4A, in some embodiments, an application provision system comprises one or more databases 200 accessed by a relational database management system (RDBMS) 210. In this embodiment, the application provision system comprises one or more web server(s) 230 and one or more application servers 220. The web server(s) optionally expose one or more web services via application programming interfaces (APIs) 240. The Application server(s) 220 contains a connector to the Smart Contract-ETH Blockchain 250. In some embodiments, the Smart Contract-ETH Blockchain 250 will be comprised of the Blockchain service 260, a Registry 280 for creating a contract with the user and the Smart Contract 270, see FIG. 4B. The application server will optionally connect to the Registry 280 at sign-up to create a new Smart Contract 270 for the user within a blockchain system 260. Examples of the blockchain system 260 include, but are not limited to, Ethereum, Hyperledger, IBM Blockchain Platform, Azure Blockchain Workbench, Amazon Quantum Ledger Database (QLDB), Kaleido Blockchain Business Cloud, and the like.

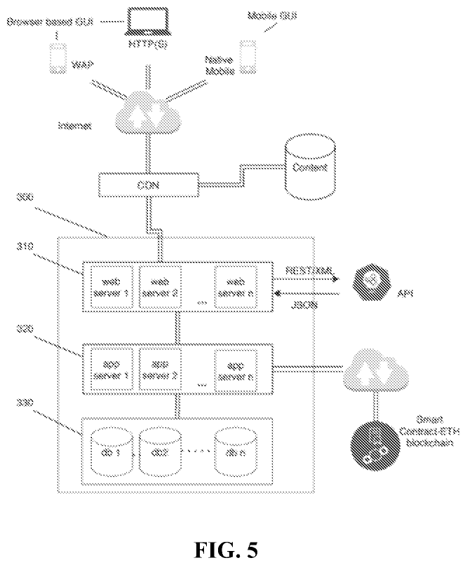

Referring now to FIG. 5, in a particular embodiment, an application provision system alternatively has a distributed, cloud-based architecture 300 and comprises elastically load balanced, auto-scaling web server resources 310 and application server resources 320, and synchronously replicated databases 330. In one example, the application provision system is an AWS cloud computing system, although other cloud-based systems may be employed. The cloud-based systems may utilize one or more services, such as for example only AWS Cognito to control access to the user pool for the sample collection kit, AWS Cloudfront to provide access to a AWS S3 repository, AWS Lamda/ECS for running code and managing resources, and AWS KMS for providing encryption. Although these examples are provided, it is to be understood that other cloud-based resources from other providers may be employed.

In some embodiments, a computer program includes a mobile application provided to a mobile computing device. In some embodiments, the mobile application is provided to a mobile computer device via the computer network (e.g., via web browser) described herein to provide a user portal for interactivity with the sample collection kit as described herein. Further, a mobile application may be provided to laboratories performing one or more analyses on the materials collected using the sample collection kit to provide a laboratory portal to communicate data related to testing of the sample collection kit.

The disclosed application provision system allows for integration between for example, a user portal and a laboratory portal, with the exemplary sample collection kit described herein. The application provision system provides two portals: (1) a user portal, for a victim to upload information and to receive testing laboratory results; and (2) a testing laboratory portal, for the laboratory to document evidence intake and to upload testing results for survivors, although the application provision system provides for other integration with the sample collection kit as described herein. For example, the application provision system allows for registration of the sample collection kit (signup), login to the application provision system for interaction therewith, sample collection kit registration, incident (activity) reporting, and image upload, by way of example, as described in further detail below, although other integrations between the application provision system and the sample collection kit are contemplated using the present technology.

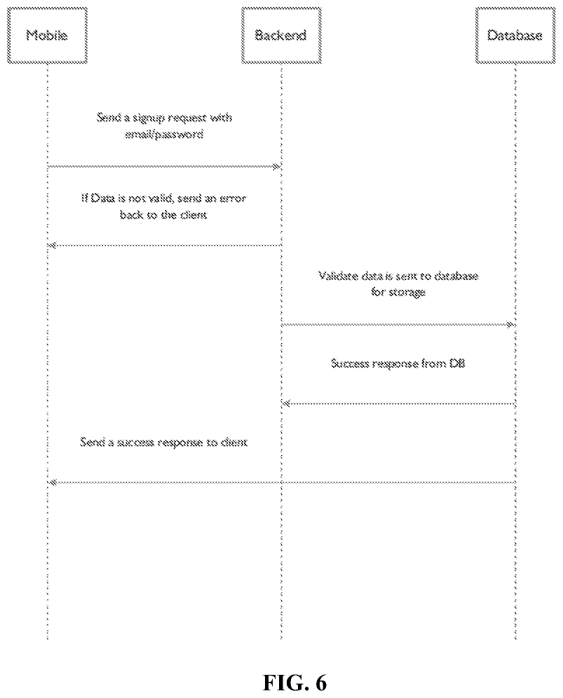

FIG. 6 illustrates an exemplary signup flow for the exemplary sample collection kit. The user may sign up for the application using, by way of example, a mobile computing device that displays a GUI from the application provision system. The user provides a signup request including the user's e-mail and password, by way of example. The application provision system (backend) verifies whether the data is valid. If not, the application provision system provides an error message back to the client (mobile) computing device. If the data is valid, the data is sent to a database associated with the application provision system for storage. In one example, the database further provides a response to the application provision system regarding successful storage of the user data as well as a response to the user (mobile) computing device to confirm a successful signup. The same signup flow may be employed to sign up a laboratory with the application provision system.

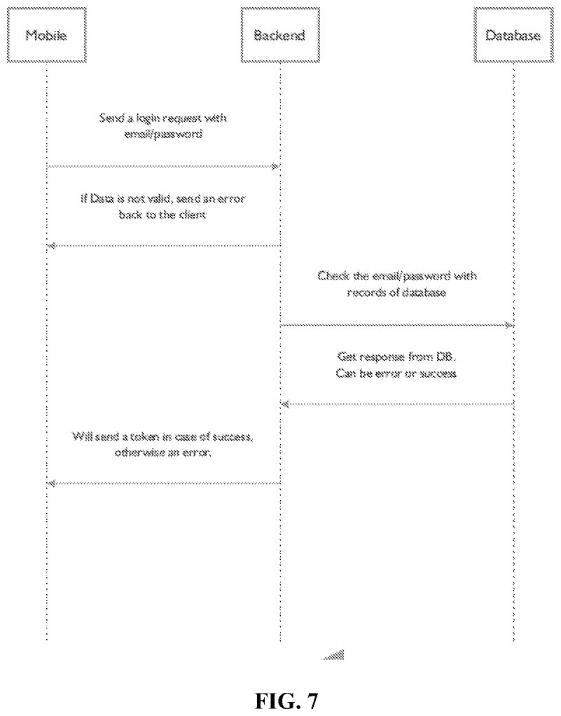

FIG. 7 illustrates an exemplary login flow for the user portal, although the same flow may be employed for the laboratory login. The user (mobile) computing device provides a login request with necessary credentials (i.e., email and password, for example). The data is validated against data stored, for example, in the database associated with the application provision system. If the data is not valid, the application provision system receives an error from the database and provides an error message back to the client. If the data is validated, the application provision system receives a validation from the database and sends a token to the user (mobile) computing device that allows for login, for example, to the user portal provided by the application provision system. The user portal allows for the user to upload information and to receive testing laboratory results, for example. The user portal in some examples also provides the user with access to customized guidance, including interactive steps and animated instructions, on use of the sample collection kit. The user portal in some examples also provides virtual access to SANEs via, for example, chat, text messages, audio calls, video calls, and file sharing (e.g., photographs).

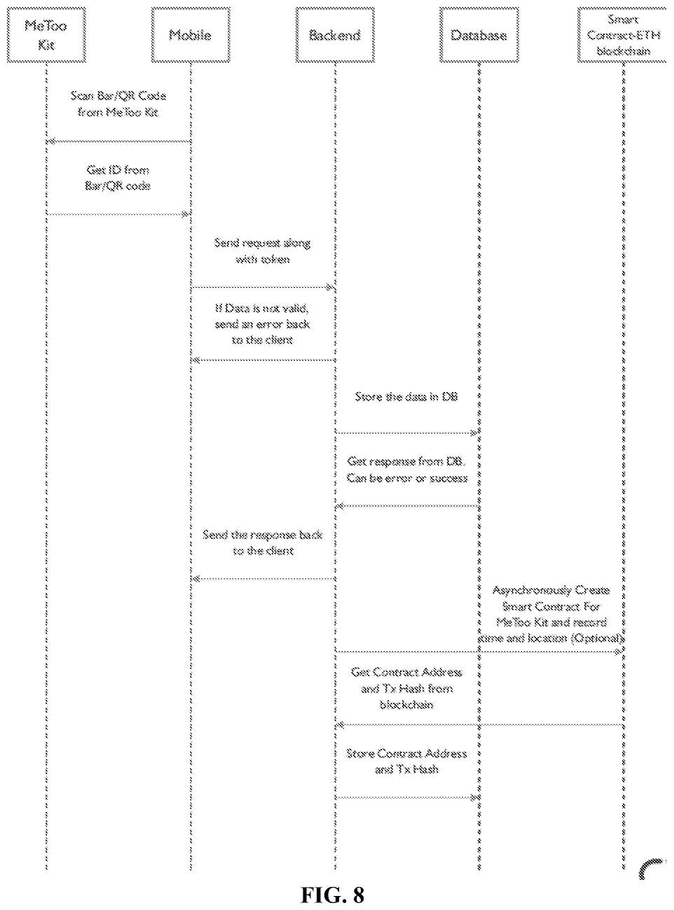

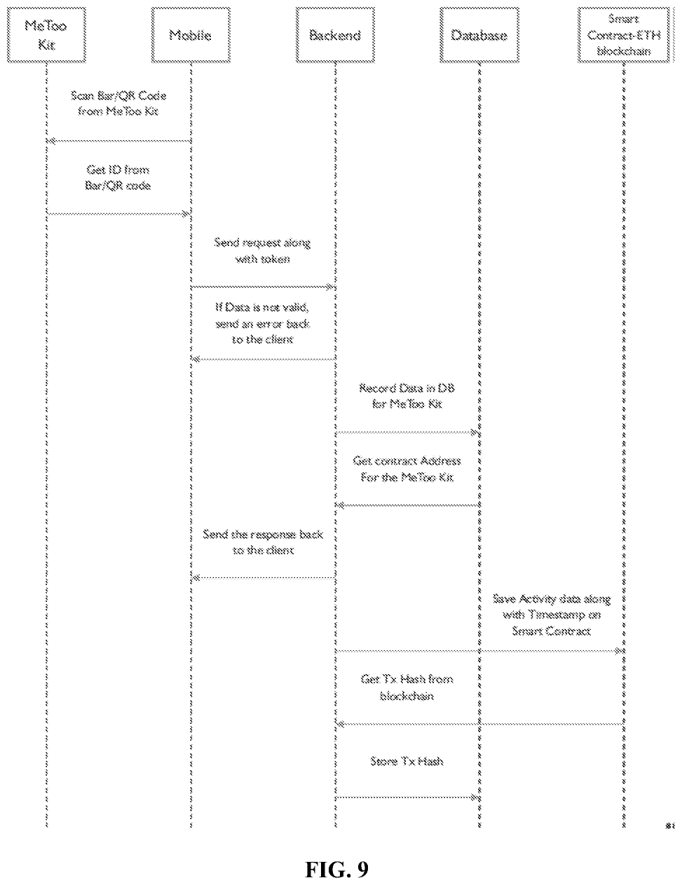

FIG. 8 illustrates an exemplary flow of kit registration for the exemplary sample collection kit (MeToo Kit). First, the user (mobile) computing device scans the unique identifier associated with the sample collection kit, such as a bar code by way of example. The unique identifier code is then obtained by the user computing device. Next, the mobile computing device provides a request along with a token to the application provision system (backend). If the data is not valid, for example if the unique identifier code has already been registered, the application provision system provides an error message to the mobile computing device. If the data is valid, i.e., the unique identifier code is valid and has not been previously registered, the application provision system stores the data, for example, in an associated database. The application provision system also asynchronously creates a smart contract for the sample collection kit using the blockchain system shown for example in FIG. 4B. This step may optionally include recording the time and location of registration of the sample collection kit, by way of example. The application provision system then receives the contact address and Tx hash from the blockchain system and stores that information in the associated database for example. Once the kit is registered, an incident, such as a sexual assault, may be reported as described in FIG. 9.

FIG. 9 illustrates an exemplary flow of incident reporting using the integrated application and sample collection kit. The materials of the sample collection kit are scanned by the user (mobile) computing device using the unique identifier codes, such as the bar codes shown in FIG. 2 for example. Next, application provision system receives a request along with a token from the user computing device. If the data is not valid, the application provision system (backend) provides an error message to the mobile computing device. If the data is valid, the application provision system stores the data for the sample collection kit, for example, in an associated database and receives a contract address from the sample collection kit from the database. The application provision system also provides a response back to the user computing device, which may be displayed, for example, on the user portal. Next, the application provision system provides activity data along with a timestamp on the smart contract to the blockchain system shown in FIG. 4B. The application provision system receives the Tx hash from the blockchain system and stores the Tx hash, for example, on the associated database.

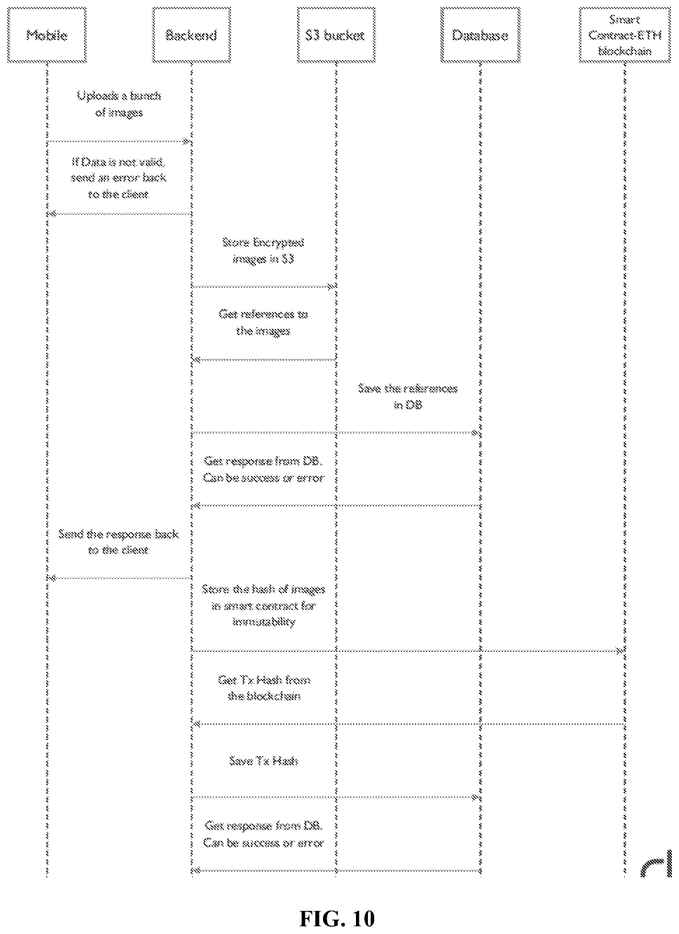

FIG. 10 illustrates an exemplary flow for image uploading using the application provision system of the present technology. Image uploading can be used to obtain evidence of, for example, sexual assault, including images of bruises or lacerations, by way of example only. First, the application provision system receives images uploaded for the user (mobile) computing device. If that data is not valid, i.e., the images cannot be properly uploaded, the application provision system provides an error message to the mobile computing device. Once the images are uploaded, the application provision system stores the images, for example, in an S3 bucket and receives references to the images, although other storage methods may be employed. The application provision system then saves the references to the images on the associated database for example and receives a response regarding whether the references to the images were successfully stored. The application provision system also provides a message to the mobile computing device regarding whether the references were successfully stored. The application provision further stores the hash of the images blockchain system shown in FIG. 4B using the associated smart contract. The application provision system receives the Tx hash for the images from the blockchain system and stores the hash in the associated database for example. The application provision system also receives a notification from the associated database as to whether or not the Tx hash is successfully saved thereon.

Although various workflows are described, it is to be understood that the application provision system can integrate with the sample collection system in other manners.

It will be apparent to the one with skill in the art that the embodiments described above are examples of a broader invention which may have greater scope than any of the singular descriptions taught. Additionally, elements of different embodiments described herein may be combined to form other embodiments not specifically set forth above.

What is claimed is:

1. A self-use sample collection kit comprising:
one or more swabs configured to collect a sample;
one or more sample collection containers configured to receive the one or more swabs; and
a unique identifier label affixed to at least a portion of the one or more sample collection containers, wherein the unique identifier label uniquely identifies the one or more sample collection containers from a population of sample collection containers, wherein the unique identifier label is linked to an integrated system for monitoring and tracking the one or more sample collection containers.

2. The sample collection kit of claim 1 further comprising:
tape configured to seal the one or more sample collection containers, the tape comprising a tamper element to identify whether the one or more sample collection containers have been opened.

3. The sample collection kit of claim 1 further comprising:
a purifying element configured to prevent or reduce contamination of the one or more swabs.

4. The sample collection kit of claim 1, wherein the one or more swabs is substantially free of DNA.

5. The sample collection kit of claim 1, wherein the one or more swabs are dry swabs.

6. The sample collection kit of claim 1, wherein the one or more swabs are sterile.

7. The sample collection kit of claim 1, wherein the one or more swabs comprise a natural fiber, a polymer fiber, or a combination thereof.

8. The sample collection kit of claim 1, wherein the one or more swabs comprise flocked fibers.

9. The sample collection kit of claim 1, wherein the unique identifier label is located on a sticker attached to the one or more sample collection containers.

10. The sample collection kit of claim 1, wherein the unique identifier label comprises a barcode, an RFID element, a QRS code, or a combination thereof.

11. The sample collection kit of claim 1, wherein the unique identifier label is configured to communicate with a digital platform or remote user.

12. The sample collection kit of claim 1, wherein the unique identifier label is configured to store data.

13. The sample collection kit of claim 1 further comprising:
an outer packaging material configured to house the sample collection kit.

14. The sample collection kit of claim 1 further comprising:
a sample bag.

15. The sample collection kit of claim 1 further comprising:
an aqueous solution.

16. The sample collection kit of claim 15, wherein the aqueous solution comprises distilled water.

17. An integrated sample collection system comprising:
the sample collection kit of any one of claims 1-16; and
an application provision system configured to provide a central application assigning an identification code to one of a plurality of the sample collection kits, the application provision system configured to:
receive an identification code related to one of the sample collection kits;
record a sample collection kit location and a timestamp;
record the sample collection kit location and timestamp in a smart contract in a blockchain system; and
receive the smart contract address and a block Tx Hash to central processor.

18. The sample collection system of claim 17, wherein the application provision system is further configured to confirm the received identification code.

19. The sample collection system of claim 17, wherein the identification code comprises a numeric code, a letter-based code, an alphanumeric code, a bar code, a QR code, an image, or any combination thereof.

20. The sample collection system of claim 17, wherein the identification code comprises a kit identification code, a container identification code, or both.
